(19) United States
(12) Patent Application Publication
(10) Pub. No.: US 2017/0143199 A1
(43) Pub. Date: May 25, 2017

(54) AIRWAY BRONCHOSCOPE

(71) Applicant: University of Utah Research Foundation, Salt Lake City, UT (US)

(72) Inventor: Johannes Fredrik Grimmer, Salt Lake City, UT (US)

(21) Appl. No.: 15/357,835

(22) Filed: Nov. 21, 2016

Related U.S. Application Data
(60) Provisional application No. 62/257,585, filed on Nov. 19, 2015.

(51) Int. Cl. A61B 1/267; A61B 1/015; A61B 17/29; A61B 1/05; A61B 1/06; A61B 1/00; A61B 1/018

(52) U.S. Cl. CPC A61B 1/2676; A61B 1/00078; A61B 1/015; A61B 1/018; A61B 1/05; A61B 1/0676; A61B 1/00137; A61B 17/29

(57) ABSTRACT

A bronchoscope system is disclosed that can include a bronchoscope and a bronchoscopy instrument. The bronchoscope can include a rigid tube for insertion into an airway of a patient. The rigid tube can have a proximal end, a distal end, and a working channel extending between the proximal and distal ends sized to facilitate ventilation of the airway via gas flow through the working channel. The bronchoscope can also include a gas port at the proximal end of the rigid tube in communication with the working channel to deliver a gas to the airway via the working channel. In addition, the bronchoscope can include an instrument port at the proximal end of the rigid tube in communication with the working channel to facilitate insertion of an instrument through the working channel into the airway. The bronchoscope can also include at least one of an illumination component and an imaging component oriented at the distal end of the rigid tube.

### RELATED APPLICATIONS

This application claims priority to U.S. Provisional Application No. 62/257,585, filed Nov. 19, 2015 which is incorporated herein by reference.

### BACKGROUND

Diseases and conditions effecting function of the tracheobronchial tree are common, can be life threatening, and often require urgent intervention. The tracheobronchial tree can be endoscopically viewed, evaluated and treated using flexible and rigid bronchoscopes. Flexible bronchoscopes are ideal for evaluating small distal airways and are used in conjunction with an established airway such as an endotracheal tube (ETT) or laryngeal mask airway (LMA). Because the flexible bronchoscope must pass inside an LMA or ETT, patient ventilation may be impaired as the bronchoscope occupies space within the airway. Flexible bronchoscopes are also limited by use of relatively smaller caliber flexible instruments, which can be more difficult to control than rigid instruments.

Rigid bronchoscopes continue to serve a vital role in endoscopic intervention of the trachea and large bronchi given their larger instrument channel compared to flexible bronchoscopes. The rigid bronchoscope also serves as an airway eliminating the necessity of an ETT or LMA. Current rigid bronchoscopes, such as the Doesel-Huzly bronchoscope (Karl Storz, Tuttlingen, Germany) use Hopkins endoscopes inserted into a central lumen of the bronchoscope. Placement of the Hopkins endoscope within the airway lumen of the rigid bronchoscope causes flow turbulence and resistance, which may contribute to complications related to ventilation. During interventions, such as foreign body removal, the endoscope must be removed, thereby opening the airway circuit, and a grasping forceps with its own endoscope (optical forceps) is inserted to remove the foreign body. Instruments without an attached endoscope (non-optical) are difficult to use because the operative field is poorly viewed. It should also be noted that the trachea is not a straight tube but has a 10-15 degree bend anteriorly as it passes below the sternal notch into the chest. Inserting a completely straight rigid bronchoscope in the trachea and bronchi requires extension of the cervical spine and puts pressure on the upper teeth, which may cause dental injuries.

Thus, some limitations of rigid bronchoscopy are increased flow resistance and turbulence within the device, use restricted to optical instruments, a complicated assembly with many parts, a non-anatomic fit, and the necessity of removing the Hopkins endoscope before inserting the appropriate optical instrument. The latter limitation is particularly problematic because during removal of the Hopkins endoscope and insertion of an instrument, the patient's airway cannot be continuously monitored and the airway circuit to the patient is opened to the surrounding environment, exposing the surgeon to anesthetic gases and ceasing flow of gases to the patient.

### SUMMARY

An inventive bronchoscope device overcomes or reduces many of these limitations. In one respect, the invention relates to establishing and maintaining an airway in a patient while evaluating and treating the tracheobronchial tree in children and adults. It will be appreciated that the same or similar apparatus or system may also be used in animals in veterinary applications.

In one aspect, a bronchoscope is disclosed that can include a rigid tube for insertion into an airway of a patient. The rigid tube can have a proximal end, a distal end, and a working channel extending between the proximal and distal ends sized to facilitate ventilation of the airway via gas flow through the working channel. The bronchoscope can also include a gas port at the proximal end of the rigid tube in communication with the working channel to deliver a gas to the airway via the working channel. In addition, the bronchoscope can include an instrument port at the proximal end of the rigid tube in communication with the working channel to facilitate insertion of an instrument through the working channel into the airway.

In another aspect, a bronchoscopy instrument is disclosed that can include an elongate structure configured to extend through a bronchoscope. The elongate structure can have an outer surface at least partially defining a width and a thickness of the elongate structure. A ratio of the width to the thickness can be greater than 1, and the outer surface can be shaped to conform to an inner surface of the bronchoscope. Although the bronchoscopy instrument can be separate from a complimentary bronchoscope, in some cases the bronchoscopy instrument can be integrated into the bronchoscope as a single unitary system.

In yet another aspect, a bronchoscope system is disclosed that can include a bronchoscope and a bronchoscopy instrument. The bronchoscope can include a rigid tube for insertion into an airway of a patient. The rigid tube can have a proximal end, a distal end, and a working channel extending between the proximal and distal ends sized to facilitate ventilation of the airway via gas flow through the working channel. The bronchoscope can also include a gas port at the proximal end of the rigid tube in communication with the working channel to deliver a gas to the airway via the working channel. In addition, the bronchoscope can include an instrument port at the proximal end of the rigid tube in communication with the working channel to facilitate insertion of an instrument through the working channel into the airway. The bronchoscopy instrument can include an elongate structure configured to extend through the working channel. The elongate structure can have an outer surface at least partially defining a width and a thickness of the elongate structure. A ratio of the width to the thickness can be greater than 1, and the outer surface can be shaped to conform to an inner surface of the working channel.

The advantages of the present system include, without limitation, improved flow of gases through the device, a more ergonomic design, increased variety of usable instruments, less assembly of parts, continuous viewing of the airway, minimized escape of anesthetic gases into the surrounding environment, and the ability to miniaturize rigid bronchoscopy for use in smaller airways (e.g. pediatric patients).

By moving an imaging and illumination system from the center of the airway throughout the length of the bronchoscope to the distal tip, there is less flow resistance and turbulence. Furthermore, with the distal end being beveled, the additional space occupied from a separate channel may not change the maximum diameter of the device nor obstruct flow of gases. In some configurations of the device, the distal tip has an in situ anterior orientation that is a more ergonomic for the anteriorly oriented trachea as is passes below the sternal notch. Flow resistance and turbulence in the design is further reduced using computer modeling software or other techniques.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 illustrates a top view of a bronchoscope in accordance with an example of the present disclosure.

FIG. 2 illustrates a side view of the bronchoscope of FIG. 1.

FIG. 3 illustrates a cross-sectional side view of the distal end of the bronchoscope of FIG. 1.

FIGS. 4A-4C illustrate cross-sectional end views toward the proximal end of the bronchoscope of FIG. 1.

FIGS. 5A-5C illustrate cross-sectional end views toward the distal end of the bronchoscope of FIG. 1.

FIG. 6A illustrates CFD results of a bronchoscope, which shows laminar flow in a gas port and proximal end of a working channel.

FIG. 6B illustrates CFD results of a bronchoscope, which shows laminar flow in a distal end of the working channel.

FIG. 7 illustrates a cross-section of a bronchoscope with certain dimensions identified.

FIG. 8 illustrates a flexible seal for an instrument channel of a bronchoscope in accordance with an example of the present disclosure.

FIG. 9A illustrates a cross-sectional side view of the distal end of the system of FIG. 1 with a different distal tip embodiment.

FIG. 9B illustrates a cross-sectional side view of the distal end of the system of FIG. 1 with a different distal tip embodiment.

FIG. 10A illustrates a cross-sectional side view of the distal end of the system of FIG. 1 with an embodiment having a cable running along the working channel in one configuration.

FIG. 10B illustrates a cross-sectional side view of the distal end of the system of FIG. 1 with an embodiment having a cable running along the working channel in another configuration.

FIG. 11 illustrates a cross-sectional side view of the distal end of the system of FIG. 1 with an embodiment having separate channels to allow cables to pass therethrough.

FIG. 12A illustrates a cross-sectional side view of the distal end of the system of FIG. 1 with at least two bevels.

FIG. 12B illustrates a cross-sectional end view of FIG. 12A.

FIG. 12C illustrates a cross-sectional top view of FIG. 12A.

FIG. 12D illustrates a cross-sectional end view similar FIG. 12C.

FIG. 13A illustrates a cross-sectional side view of the distal end of the system of FIG. 1 with at least three bevels.

FIG. 13B illustrates a cross-sectional end view of FIG. 13A.

FIG. 13C illustrates a cross-sectional top view of FIG. 13A.

FIG. 13D illustrates a cross-sectional end view similar FIG. 13C.

FIG. 14 illustrates a cross-sectional side view of the distal end of the system of FIG. 1 with an embodiment having the inside wall of a hardware channel tapering toward a central axis of the working channel.

FIG. 15 illustrates a cross-sectional side view of the distal end of the system of FIG. 1 with an embodiment having a distal tip using wireless technology.

FIG. 16 illustrates a bronchoscope system in accordance with an example of the present disclosure with an instrument configured as a catheter guide.

FIGS. 17A-17C illustrate cross-sectional views of the bronchoscope system of FIG. 16.

FIG. 18 illustrates a bronchoscope system in accordance with another example of the present disclosure with an instrument configured as forceps.

FIG. 19 illustrates a cross-sectional view of the bronchoscope system of FIG. 18.

FIG. 20 illustrates a cross-section of the forceps instrument within a bronchoscope, with width and thickness dimensions identified for an elongate structure of the forceps.

FIG. 21 illustrates a bronchoscope system in accordance with another example of the present disclosure with an instrument configured as a catheter forceps.

FIG. 22 illustrates a cross-sectional view of the bronchoscope system FIG. 21.

FIG. 23 illustrates a cross-section of the catheter forceps instrument within a bronchoscope, with certain dimensions identified for the instrument.

FIG. 24 illustrates a bronchoscope system having integrated forceps in accordance with yet another example of the present disclosure.

FIG. 25 illustrates a cross-section of the bronchoscope system of FIG. 24.

FIG. 26 is a graph of hemodynamic stability over time for animals subjected to mechanical ventilation using a control bronchoscope and an airway bronchoscope.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

Definitions

In describing and claiming the present invention, the following terminology will be used.

The singular forms “a,” “an,” and “the” include plural referents unless the context clearly dictates otherwise. Thus, for example, reference to “a surface” includes reference to one or more of such surfaces and reference to “the instrument” refers to one or more of such instruments.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, “rigid tube” refers to a tube having sufficient structural rigidity to be guided into bronchi branches by direct manipulation by the operator. Typically, the rigid tube can be formed of stainless steel or other metal, although a rigid plastic can also be used as long as elasticity is limited to avoid a compliant or bendable device which would require the distal tip to be controlled remotely for directional control. Rigid tubes are in contrast to flexible scopes and tubes which are guided into the branches of the bronchi by controlling the distal tip with a hand control.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Bronchoscope

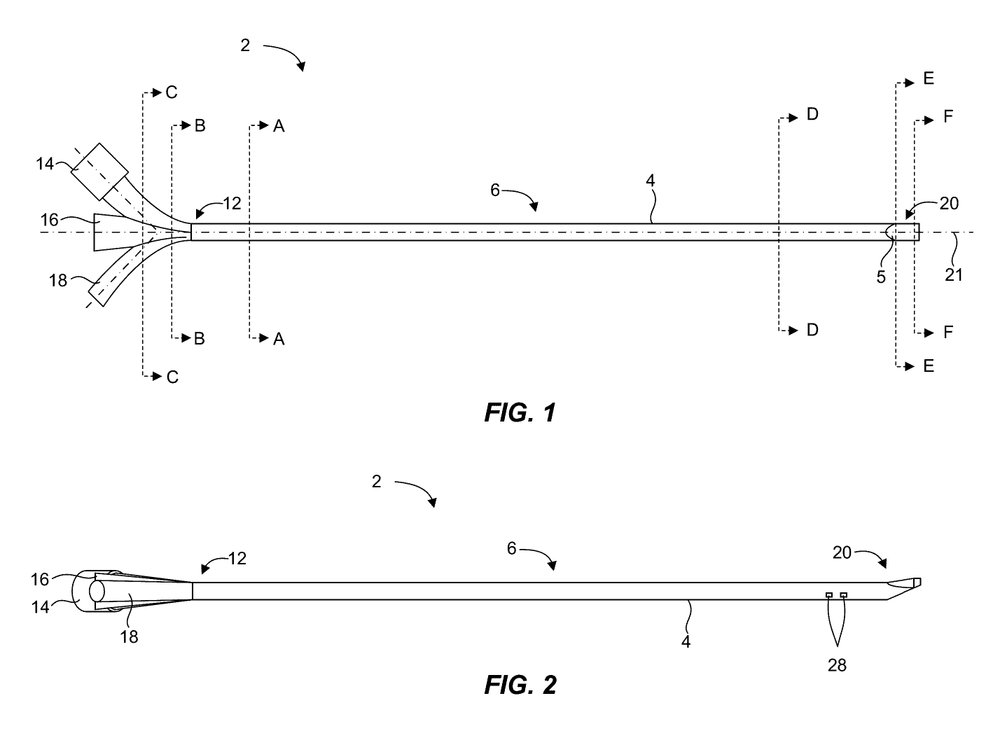

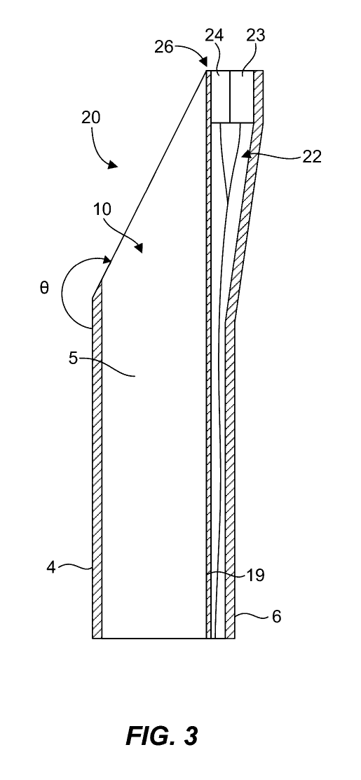

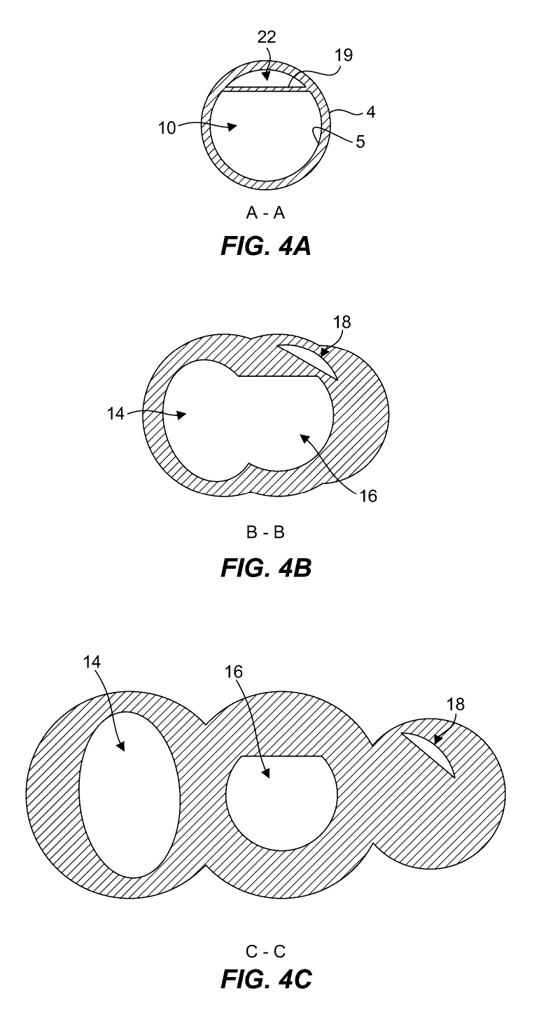

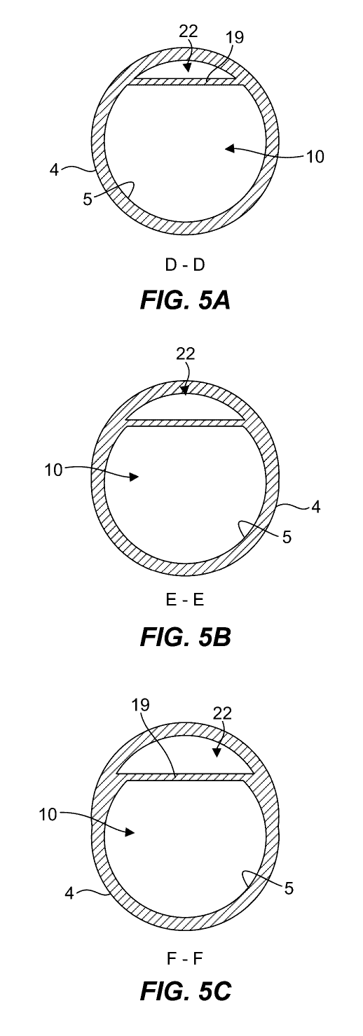

FIGS. 1-5C illustrate various aspects of a bronchoscope 2 in accordance with an example of the present disclosure. FIGS. 1 and 2 illustrate top and side views, respectively, of the bronchoscope 2. FIG. 3 illustrates a detailed view of a tip or distal end of the bronchoscope. FIGS. 4A-4C illustrate cross-sectional views of a proximal end of the bronchoscope 2, and FIGS. 5A-5C illustrate cross-sectional views of the distal end of the bronchoscope 2.

With particular reference to FIGS. 1-3, the bronchoscope 2 can include a rigid tube 6 for insertion into an airway of a patient. The rigid tube 6 can have a proximal end 12, a distal end 20, and a working or main channel 10 extending between the proximal and distal ends sized to facilitate ventilation of the airway via gas flow through the working channel. The working channel 10 of the tube 6 can be referred to herein as a lumen or conduit. The rigid tube 6 can also include a hardware channel 22, which can run adjacent to and parallel with the working channel 10. The working channel 10 and the hardware channel 22 can be separated by an inner or separating wall 19.

In one aspect, the bronchoscope 2 can include an illumination component 23 (e.g., a light) and/or an imaging component 24 (e.g., a camera) at the distal end 20 of the rigid tube 6, which can be positioned within an outer wall 4 of the rigid tube 6. For example, the illumination component 23 and/or the imaging component 24 can be disposed or fixed at the distal end 20 of the hardware channel 22. The imaging system 24 and illumination source 23 can reside within the hardware channel 22 or hardware chamber. The illumination component can be oriented along a side of the imaging system, oriented about either side, as a ring COB LED, stacked longitudinally, or other suitable orientation. The hardware channel 22 can widen distally to house the camera 24 and/or the illumination source 23. In one aspect, the hardware channel 22 can extend to a distal tip 26 and encompass the camera 24 and illumination source 23 partially or entirely with the outer wall 4 and an inner wall 19. The rigid tube 6 can be beveled at the distal end 20 to increase the opening size of the working channel 10 and reduce the overall dimension of the distal end 20.

The bronchoscope 2 can include various ports at the proximal end 12 of the rigid tube 6. For example, the bronchoscope 2 can include a gas or anesthesia port 14, an instrument port 16, and a hardware port 18. The gas port 14 and the instrument port 16 can be in communication with the working channel 10 to deliver a gas to the airway via the working channel. Although the ports are illustrated in a particular orientation, variations in design can be made while retaining performance of the device. For example, the hardware port 18 is illustrated as extending from a side of the working channel 10. However, the hardware port 18 can also be oriented at a top of the bronchoscope at the proximal end.

The gas port 14 can connect to an anesthesia machine via anesthesia tubing. Anesthesia can therefore be delivered to the patent's airway via the gas port 14, which communicates directly with the working channel 10. The gas port 14 can be oriented away from the proximal end 12 of the rigid tube 6 at an angle less than 90 degrees relative to a longitudinal or central axis 21 of the rigid tube 6. In one aspect, the gas port 14 can include a gentle curve and can extend from the outer wall 4 of the rigid tube 6 at an acute angle relative to a longitudinal axis 21 between the anesthesia port 14 and proximal end 12. The instrument port 16 can also communicate directly with the working channel 10 to pass instruments into the airway. The instrument port 16 can be aligned or oriented parallel to the longitudinal axis 21 of the rigid tube 6. In one aspect, the outer walls of the instrument port 16 can be aligned parallel with the working channel 10 or funnel outward to provide a larger opening to ease instrument insertion. The working channel 10 can therefore serve the dual purpose of facilitating ventilation of the airway and providing a channel or conduit to facilitate insertion of an instrument into the airway. The hardware port 18 can be in communication with the hardware channel 22 to facilitate communication with the illumination component 23 and/or the imaging component 24. The hardware port 18 can be oriented at any suitable angle relative to the longitudinal axis 21 of the rigid tube 6. The gas port 14, the instrument port 16, and the hardware port 18 can be oriented in a common plane (as shown in FIG. 2) or in any other suitable orientation. It should be recognized that any other suitable port can also be included at the proximal end 12 of the rigid tube 6, such as a port for passing flexible instruments, catheters, laser fibers, or other instruments. The ports can allow a surgeon appropriate workspace and can be integral to the body of the device and provide ease of use without assembly of multiple parts to allow function of the device.

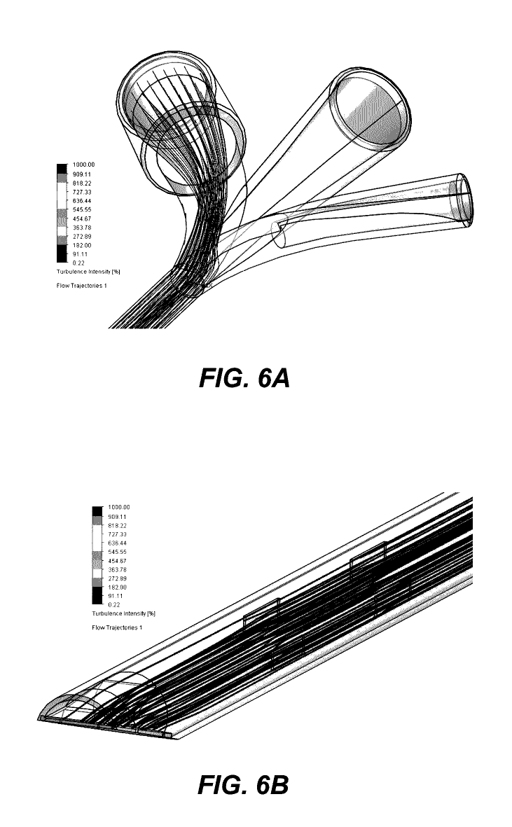

FIGS. 4A-5C demonstrate how gases can flow from the anesthesia port 14 into the working channel 10 and then out of a distal end 20. The flow of gases may occur in an elliptical to round space with a slight flat surface from an inner wall 19 of a hardware channel 22. As shown in the figures, a cross-sectional shape of an inner surface 5 of the working channel 10 is at least partially rounded. The imaging component 24 and/or the illumination component 23 can be located adjacent to the wall of the bronchoscope rather than occupying space in the central lumen which creates flow resistance and turbulence. Using computer modeling software or other technology, turbulence and flow resistance can be reduced throughout the gas port 14 and the working channel 10. For example, the gentle curve of the gas port 14 that extends from the sidewall between the gas port 14 and proximal end 12 can be configured to improve gas flow. In one aspect, the structural configuration of the gas port 14 transitioning to the working channel 10 can be such that gas flow is laminar between the gas port 14 and the working channel 10. Such structural configurations can be achieved by utilizing computational fluid dynamics (CFD), which uses numerical analysis to analyze fluid mechanics. FIGS. 6A and 6B illustrate CFD results of the bronchoscope, which shows laminar flow in the gas port and proximal end of the working channel (FIG. 6A) and laminar flow in the distal end of the working channel (FIG. 6B). In particular, the CFD results show a favorable distal exit pattern, with a majority of the flow directed to the distal opening or airway. Thus, the gas port 14 and the working channel 10 can be configured to maintain laminar flow through the port and channel, which results in better gas flow and ventilation of the airway.

FIGS. 4A-4C demonstrate how the hardware channel 22 transitions to the hardware port 18. The hardware channel 22 and hardware port 18 do not communicate with the working channel 10 in the primary embodiment. The inner wall 19 of the hardware channel 22 may be flat and smooth to minimize flow resistance and turbulence. The inner wall 19 (e.g. see FIG. 4A) can have a planar shape or the inner wall can be curved. For example, the inner wall may have a slight concave curvature with respect to the working channel 10 such that flow disturbance is further minimized. The distal end 20 of the hardware channel 22 houses the camera 24 and illumination source 23. At the distal end, in a distal to proximal direction, the hardware channel 22 funnels down to a smaller space to accommodate imaging and illumination components, such as fiber optics (i.e., illumination from an external light source) and wiring, which require less space, providing a large working channel 10. Greater description and detail is further outlined herein.

As shown in FIG. 3, distal ends of the inner wall 19 and the outer wall 4 can be nonparallel in a direction along the longitudinal axis 21 of the rigid tube 6. In this example, the distal end of the outer wall 4, which can be the outer wall of the hardware channel 22, is angled away from the longitudinal axis 21, and the distal end of the inner wall 19 is straight in the direction along the longitudinal axis 21. In this configuration of the device, the working channel 10 can accommodate large caliber rigid instruments because the inner surface 5 of the working channel 10 are parallel. In addition, the distal tip 26 is effectively angled away from the central axis 21 of the working channel 10. Thus, by beveling the tip and angling the outer wall 4 of the hardware channel 22 (i.e., of the rigid tube 6) away from the central axis 21 of the bronchoscope 2, the distal end 20 is effectively angled anteriorly providing a more anatomic fit as the bronchoscope passes inferiorly within the trachea below the sternal notch into the chest. For demonstration purposes, the dimensions for a specific pediatric-sized trachea can measure 6 mm in cross-sectional diameter. The system can come in various sizes to fit pre-term infants up to adults. Dimensions and bevel angle for any specific size of the system can vary depending on wall thickness, size of hardware channel 22 and other variables. The distal end 20 of the working channel 10 can be beveled to increase the size of the distal opening and reduce the maximum diameter of the distal tip, which can provide improved airflow and ease insertion of the bronchoscope. The outer wall 4 of the tube 6, which can be the outer wall of the hardware channel 22, angles away from the central axis 21 creating a large space to house the camera 24 and illumination element 23 (illumination source or light source such as fiberoptics). With present technology, the widest part of the imaging system is a digital sensor and the wiring is negligible. Present technology can allow placement of a 1×1×2 mm CMOS sensor (Owaiba, Funchal, Madeira) at the distal end 20. The wall of the working channel 10 can be manufactured using 3-D printing technology or other techniques. Wall thickness of the working channel 10 can be made as thin as 0.1 mm using stainless steel, or other similar materials while maintaining sufficient strength. Those other materials can include polymers and other metals, which can allow thinner wall thickness. Wall thickness can vary depending on size of the bronchoscope 2 (FIGS. 1-2) and desired strength of the tube 6.

The dimensions and bevel angle can vary depending on the size of the bronchoscope 2 (FIGS. 1-2), size of the camera 24, size of the hardware channel 22, and wall thickness of the working channel 10. Generally, a bevel can be used to reduce cross-sectional area at the distal tip when the hardware channel flares out, while also maintaining fluid flow. In one aspect, the bevel can start near a longitudinal location 23 where the hardware channel 22 begins to flare outward (e.g. see FIG. 3). The bevel angle (θ) is measured, in a side view configuration, by measuring the angle created from the bottom wall of the working channel 10 to the most distal point on the top wall of the working channel 10. For example, in FIG. 3 the bevel angle θ is 198 degrees. The desired bevel angle can be dependent on a ratio between the diameter and length of the hardware channel 22 and the diameter of the working channel 10, although a range of bevel angles (e.g. 181-269 degrees) can work for any specific device. Bevel angles from 190 to 248 degrees are particularly useful, although desired bevel angles can depend on a balance of excessive sharpness, which can pierce tissue, with bluntness, which can cause trauma to tissue during insertion. The smaller the ratio, the greater the upper limit of the bevel angle. The larger the said ratio, the smaller the bevel angle needed to create a long tapered distal end 20. The lower limit of the bevel angle is greater than 180 degrees when the maximum dimension of the hardware channel 22 approaches the diameter of the working channel 10. Desired bevel angles can also vary depending on cross-section profiles at the tip. For example, a diagnostics camera tends to be larger (e.g. greater than about 2 mm) such that a much longer bevel (e.g. lower bevel angle θ) to avoid exceeding max dimensions, while a smaller camera such as recently developed 1 mm chips or smaller can allow for a larger bevel angle θ which in some cases would then only be limited by tissue damage considerations.

This bronchoscope 2 can also allow further miniaturization of rigid bronchoscopy technology. The smallest available rigid bronchoscope (Karl Storz, Tuttlingen, Germany) has an outer diameter of 4 mm (2.5 conventional sizing) employing a 1.9 mm cross-sectional diameter Hopkins endoscope. Further miniaturization is limited by the space for the airway between the inner surface of the bronchoscope and the outer wall of the Hopkins endoscope. As mentioned, the present embodiments avoid these limitations by housing the imaging and illumination system in the distal end 20 and directing imaging and illumination components in the small hardware channel 22 adjacent to the working channel 10. For preterm infants with 3 mm cross-sectional diameter tracheas, the bronchoscope 2 could be made with a maximum outer diameter of 2.5 mm using a wall thickness of 0.1 mm and 1×1×2 mm CMOS sensor (Owaiba, Funchal, Madeira) while maintaining a 2 mm internal diameter of the working channel 10. While flexible bronchoscopes are available in sub 3 mm sizes (Olympus, Tokyo, Japan), they do not have any instrument channels in these sizes.

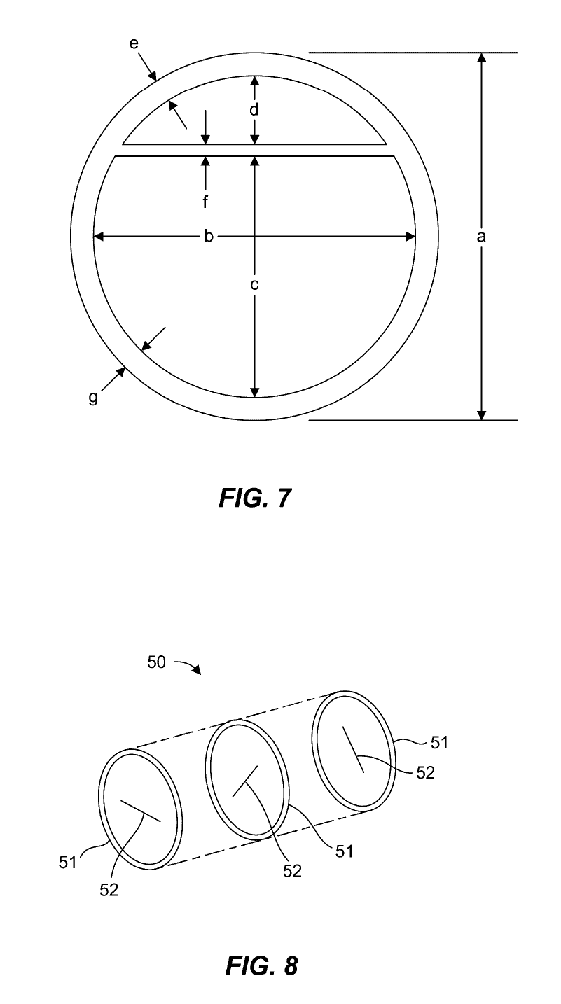

The rigid tube 6 of the bronchoscope 2 can have a length and diameter appropriate for various patient sizes. The rigid tube 6 can be any smooth hollow cross-sectional shape such as circular, elliptical, oval or polygonal shape. The outer diameter can range from 2 to 25 mm and the length can range from 3 to 150 cm. FIG. 7 illustrates a cross-section of a bronchoscope with certain dimensions identified. Examples of suitable values for these dimensions are found in Table 1, below.

TABLE 1

| | | <30 Weeks | Newborn/ | 0-12 | 12-24 | 2-4 | >4 |
|---|---|---|---|---|---|---|---|
| ID | Description | Premature | Premature | Months | Months | Years | Years |
| a | Ideal O.D. (mm) | 3.6 | 4.0 | 5.0 | 5.7 | 6.4 | 6.7 |
| a | Range of O.D. (mm) | 2.0-3.9 | 3.7-4.9 | 4.1-5.6 | 5.1-6.3 | 5.8-6.6 | 6.5-7.5 |
| b | Ideal Working Channel | 3.0 | 3.4 | 4.4 | 5.1 | 5.8 | 6.1 |
| | width (mm) | | | | | | |
| b | Working Channel width | 0.1-3.8 | 3.1-4.9 | 3.3-5.6 | 4.3-6.3 | 5.2-6.6 | 5.7-7.4 |
| | (mm) | | | | | | |
| c | Ideal Working Channel | 2.1 | 2.5 | 3.5 | 4.2 | 4.9 | 5.2 |
| | Height (mm) | | | | | | |
| c | Working Channel Height | 0.1-3.2 | 2.0-3.6 | 2.6-4.6 | 3.6-5.3 | 4.3-6.0 | 5.0-7.4 |
| | (mm) | | | | | | |
| d | Ideal Hardware Channel | 0.7 | 0.7 | 0.7 | 0.7 | 0.7 | 0.7 |
| | Height (mm) | | | | | | |
| d | Hardware Channel | 0.1-1.0 | 0.1-1.5 | 0.1-2.0 | 0.1-2.5 | 0.1-3.0 | 0.1-3.5 |
| | Height (mm) | | | | | | |
| e | Ideal Hardware Channel | 0.3 | 0.3 | 0.3 | 0.3 | 0.3 | 0.3 |
| | Wall Thickness (mm) | | | | | | |
| e | Hardware Channel Wall | 0.1-0.6 | 0.1-0.6 | 0.1-0.6 | 0.1-0.6 | 0.1-0.6 | 0.1-0.6 |
| | Thickness (mm) | | | | | | |
| f | Ideal Partition Wall | 0.2 | 0.2 | 0.2 | 0.2 | 0.2 | 0.2 |
| | Thickness (mm) | | | | | | |
| f | Partition Wall Thickness | 0.1-0.6 | 0.1-0.6 | 0.1-0.6 | 0.1-0.6 | 0.1-0.6 | 0.1-0.6 |
| | (mm) | | | | | | |
| g | Ideal Working Channel | 0.3 | 0.3 | 0.3 | 0.3 | 0.3 | 0.3 |
| | Wall Thickness (mm) | | | | | | |
| g | Working Channel Wall | 0.1-0.6 | 0.1-0.6 | 0.1-0.6 | 0.1-0.6 | 0.1-0.6 | 0.1-0.6 |
| | Thickness (mm) | | | | | | |

The construction of the bronchoscope 2 can be made from any material, including biocompatible material which provides sufficient strength to wall thickness, such as stainless steel, titanium, cobalt chrome, a polymer or the like. The bronchoscope 2 can also be comprised of various materials within the same embodiment such as the working channel 10 of the tube 6 made of stainless steel and the proximal end with the multiple ports 14, 16, 18 comprising a rigid polymer. The wall 4 thickness of the tube 6 can be made as thin as possible to maximize the inner diameter while maintaining appropriate strength. The bronchoscope 2 can be manufactured using 3-D printing technology or any other suitable manufacturing technique.

In one aspect, the instrument port 16 can be sealed unless passing an instrument. Various seals can be used for the instrument port 16 including but not limited to hinged doors, sliding doors, caps, valves, covers or a membrane system. In one aspect, a membrane system associated with the instrument port 16 can allow penetration of instruments with minimal gas escape, as utilized in a Bodai adaptor (Sontek Medical, Inc., Hingham, Mass., USA). Such a flexible seal 50 is illustrated in FIG. 8 and can self-seal about an instrument when inserting the instrument through the working channel 10 into the airway. The seal 50 can include one or more flexible diaphragms 51 or membranes that have an opening 52, which can be defined by a slit or slice. The slit can be any suitable shape such as, but not limited to, straight, S-shaped, curved (arc), or curvilinear. A diaphragm assembly can include multiple flexible diaphragms 51 in series. The slit openings 52 can be arranged so that each slit is angularly displaced or offset (e.g., about sixty degrees from the slits on the adjacent diaphragms). This offset of the slit openings 52 can aid in maintaining a pressure maintainable seal while inserting and removing an instrument. The diaphragms 51 can be mounted close together to insure a tight seal is maintained. Although each illustrated diaphragm 51 includes only a single slit opening 52, it should be recognized that a diaphragm can include multiple slits (e.g., angularly offset from one another). The diaphragms can be made of any suitable material, such as latex. A flexible diaphragm or membrane seal can seal a variety of instrument types, shapes, and sizes and retains its sealing ability throughout the insertion and removal of the instruments. The instrument port 16 can therefore be sealed to maintain a closed airway circuit. The operator can pass instruments through the instrument port 16, which can be self-sealed with minimal gas escape when passing instruments. Once the procedure is completed, the flexible diaphragm seal can be discarded and replaced with a new seal.

The imaging system 24, which can include a camera or other suitable sensor, has various focal lengths and camera angles depending on the particular size of the device and application. With the camera being off-center of the bronchoscope's 2 central axis 21, for most applications the camera angle can be directed such that the device or distal tip is centrally positioned within the tracheobronchial tree when the image is centered at the lumen of the tracheobronchial tree. Maintaining the bronchoscope 2 in the center of the tracheobronchial tree is necessary to avoid trauma to the mucosal surfaces on insertion. The camera angle varies depending on the focal length and off-center distance of the camera. A typical focal length can range from 1 to 50 mm. The camera angle can be determined with the off-center distance and focal length using the Pythagorean Theorem or other methods to optimize viewing. Alternatively, in specific applications the camera angle could be directed to the sides of the airway to provide better viewing of the mucosal surfaces and distal bronchi. An adjustable camera angle with proximal control could also be valuable for examining distal bronchi as they branch off from the main stem bronchi. The bronchoscope 2 may not be limited to one camera. Multiple cameras can be utilized with the bronchoscope 2, either placed side by side or at a distance apart, using, but not limited to, the various configurations described herein.

There are many options for imaging and is not limited to CMOS sensor technology. The imaging system can be as small as possible with an acceptable image, thus maximizing laminar flow at the distal end 20 of the working channel 10. The wiring of the imaging system can run in the hardware channel 22, and exits via the hardware port 18, ultimately connecting to a processor, such as a computer and/or monitor. Alternatively, the hardware of the imaging and illumination system could also be fixed to the inner surface 5 of the tube 6 or outer wall 4 without a separate hardware channel 22 (see, e.g., FIGS. 10A, 10B, and 15) or incorporated into the wall of the working channel 10. The inner wall 19 separating the hardware channel 22 and working channel 10 could be made of a different thinner material that does not, necessarily, provide significant structural support to the device.

The lighting can emanate from the distal end 20. There are many options for illumination including but not limited to low-heat emitting lights bulbs, LED, fiber optics with an external light source, or luminescent materials. In some versions of the present embodiment, the wiring or fiber optics from the illumination system 23 can run adjacent to the wiring of the imaging system 24 and exit via the hardware port 18 ultimately connecting to a power or light source.

Within the body of the first chamber 10, the cabling for illumination and imaging require minimal space and run in the narrow hardware channel 22 or are fixed to the wall of the working channel 10. By utilizing thin walls in the working channel 10 and a narrow hardware channel 22, the tube 6 of the bronchoscope 2 has a relatively large interior diameter with a similar external diameter as other standard or similar sized ETTs.

Furthermore, by having a fixed illumination source 23 and imaging system 24 at the distal end 20 provides continuous monitoring of the airway without need for Hopkins or similar endoscopes. Despite the orientation of the distal end 20, in some versions of the present embodiment, the inner surface 5 of the working channel 10 is maintained straight in a direction along the longitudinal axis 21 to facilitate passage of rigid instruments. This is important because straight rigid instruments generally offer more fine control over flexible instruments. This also opens the door to a greater variety of instruments including, but not limited to, powered instruments such as coblators (ArthroCare, Austin, Tex., USA), microdebriders (Stryker, Kalamazoo, Mich., USA), and stapling devices, which are not available as optical instruments. These devices, as well as many others, have pioneered minimally invasive surgery in other fields and may prove useful within the tracheobronchial tree.

The present embodiments contemplated herein require less assembly for proper functioning eliminating the need for placement of endoscopes and attachment of cameras, a valuable feature in an emergent setting such as foreign body aspiration. The devices contemplated herein also have a less complicated system for instrument insertion compared to conventional rigid bronchoscopes, thereby reducing the time the airway circuit is opened, which exposes the surgeon to anesthetic gases and limits flow of gases to the patient.

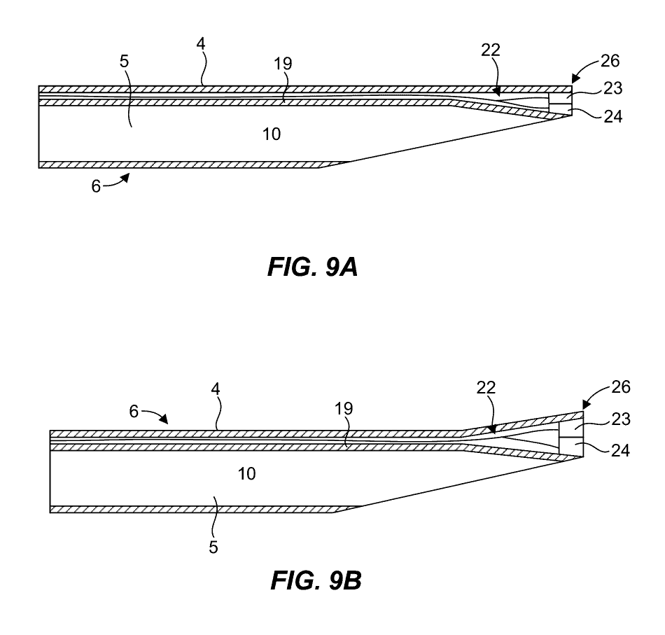

Referring to FIGS. 9A and 9B, alternate embodiments of the bronchoscope 2 are contemplated with alternate configurations of the distal end 20 of the hardware channel 22. In FIG. 9A, the distal end of the inner wall 19 (forming the hardware channel 22) is angled toward the longitudinal axis 21 of the working channel 10, and the distal end of the outer wall 4 is straight in the direction along the longitudinal axis 21. This configuration of the bronchoscope 2 maintains a straight outer wall 4 of the working channel 10, which can provide for a uniform outer dimension of the rigid tube 6 along its length. In FIG. 9B, the outer wall 4 of the hardware channel 22 is angled away from the longitudinal axis 21 of the working channel 10 and the inner wall 19 of the hardware channel 22 is angled toward the longitudinal axis 21 of the working channel 10. In this configuration, the hardware channel 22 has increased in dimension without increasing the maximum cross-sectional dimension of the device compared to the example shown in FIG. 3. The distal tip 26 is angled away from the central axis 21 of the working channel 10. This latter configuration can allow placement of a relatively larger camera without distal obstruction of gases and without increasing the cross-sectional dimension of the device compared to some other embodiments. Alternatively, in some embodiments, distal ends of the inner wall 19 and the outer wall 4 can be parallel in a direction along the longitudinal axis 21 of the rigid tube 6.

As previously set forth herein, FIGS. 10A and 10B contain side sectional views of the distal end 20 of the device with alternate configurations. In FIG. 10A, the cables for imaging and illumination, such as fiber optics or wiring, run along the inner surface 5 of the working channel 10 without an inner channel 22. FIG. 10B illustrates the cables running along the outer wall 4 of the working channel 10. For both alternate configurations in FIGS. 10A and 10B, the cables could be permanently or removably fixed to the wall of the working channel 10 or remain free.

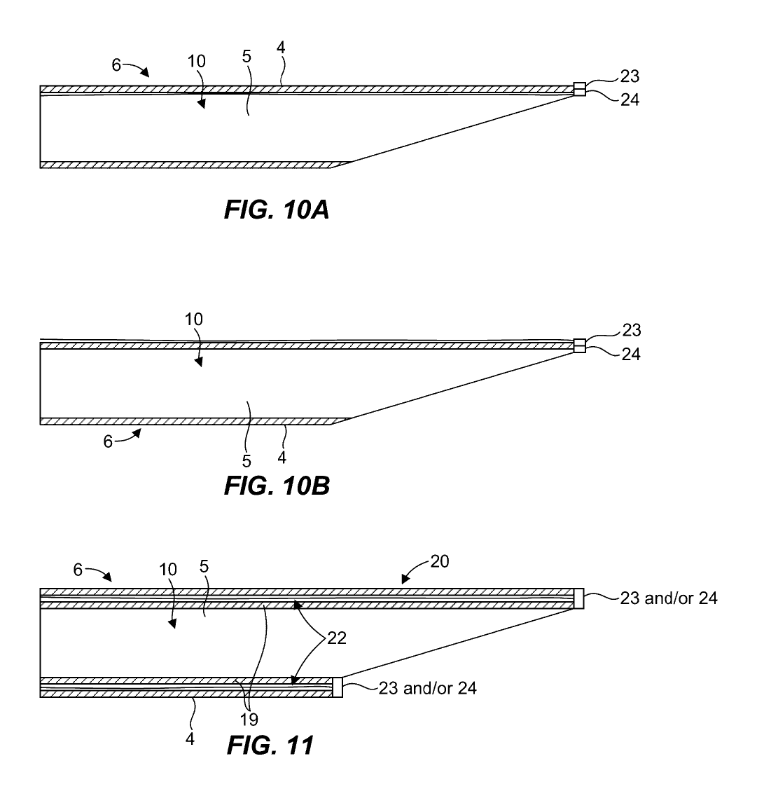

Alternatively, FIG. 11 depicts multiple inner channels 22 wherein the illumination source 23 and imaging system 24 can be in separate channels. It will also be appreciated that multiple cameras and multiple imaging sources can be placed in each channel. In this particular embodiment, the camera 24 and illumination source 23 can be fixed to the distal end 20. This alternate configuration of the device can improve space optimization and reduce glare by keeping the illumination source 23 and imaging system 23 further apart.

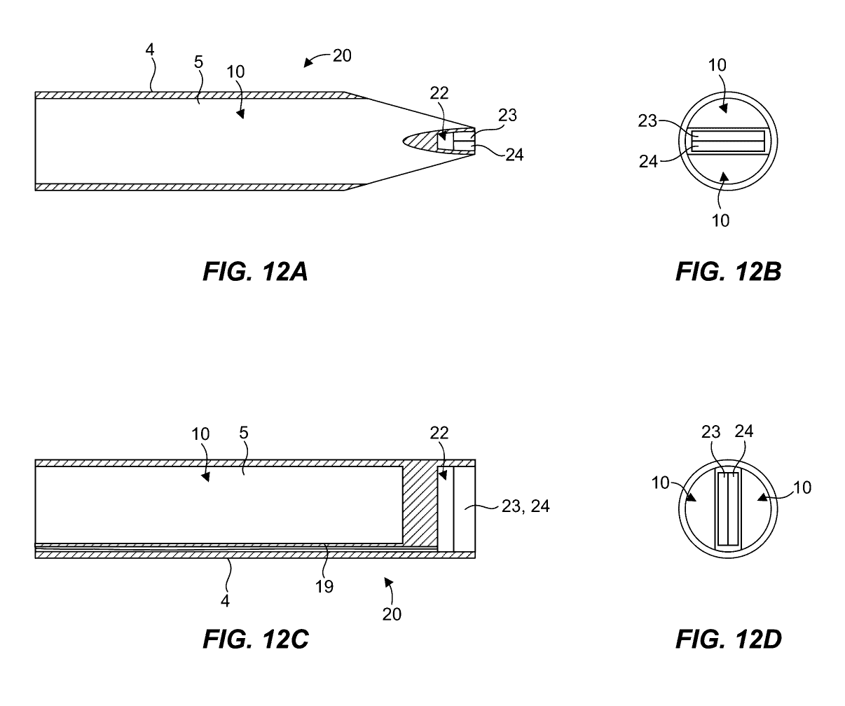

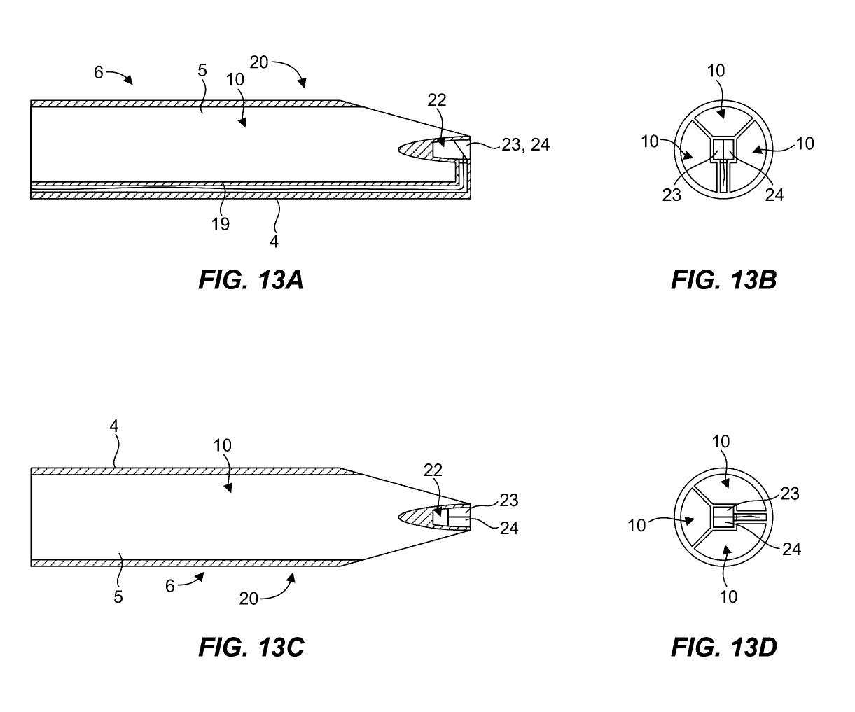

Referring to FIGS. 12A-12D, another embodiment can include the bronchoscope 2 with a bi-beveled tip. In this configuration, the camera 24 and illumination source 23 can be centered at an axial plane of the working channel 10. This alternate configuration can allow placement of a larger camera 24 or placement of two or more cameras. This alternate configuration of the bronchoscope 2 favors imaging quality over the ability to pass large caliber rigid instruments in the working channel 10. Alternatively, FIGS. 13A-13D depict the bronchoscope 2 with a tri-beveled tip. The number of bevels can vary depending on the application. The use of multiple bevels can keep the camera aligned within the central axis 21 of the working channel 10, which can make handling easier. Placing multiple bevels on the distal end of the device creates multiple large distal openings of the working channel and directs gases obliquely around the camera 24 and illumination source 23. The distal openings of the working channel 10 are oriented obliquely and can limit instrument use to small caliber rigid instruments or flexible instruments.

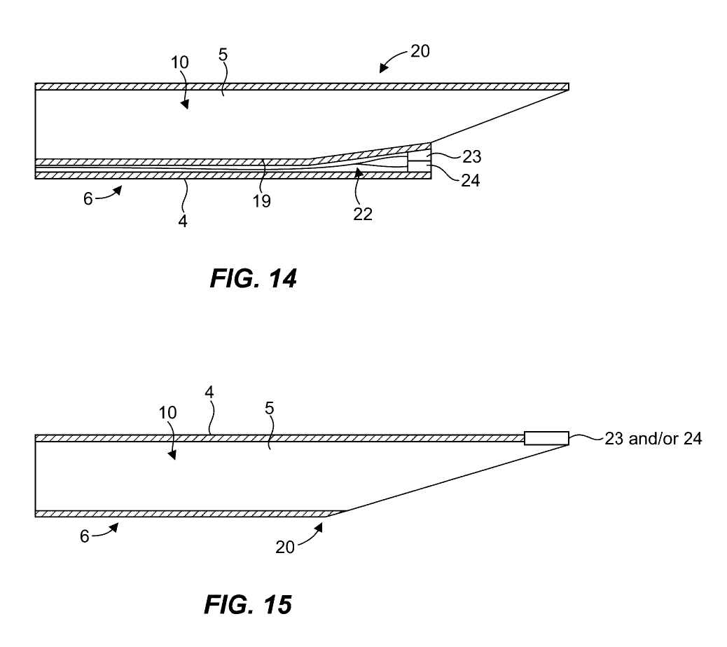

In each of the previous embodiments it is contemplated that the hardware channel 22 can run along the long side of the tube 6 to the distal tip 26. However, it is further contemplated that the inner channel can run along the shorter side of the tube 6, as shown in FIG. 14. In this configuration, the hardware channel 22 decreases the distal opening, obstructing flow of gases and limiting use to small caliber instruments because the inner wall 19 is angled toward the central axis 21. The outer wall 4 of the device at the distal end 20 are parallel and straight. The inner wall 19 and the outer wall 4 at the distal end 20 of the device are not parallel. The distal tip 26, however, provides a hood over the imaging system 24 and illumination source 23 which protects the camera 24 and illumination source 23 from surrounding secretions. The tip extends distally beyond the imaging 24 and illumination source 23 preventing direct tissue obstruction. This alternate configuration of the device can be valuable when there are copious sections or blood within the tracheobronchial tree protecting the imaging 24 and illumination source 23 from becoming obscured. This alternate configuration may be of value if a forceps or other instrument is fixed to the distal tip, providing the optimal viewing location for the camera and maintaining the working jaws of the forceps at the most distal location. In other embodiments, the imaging system 24 and illumination source 23 can utilize wireless technology, which can eliminate the hardware channel from the bronchoscope, as illustrated in FIG. 15.

In each of the embodiments disclosed herein it is conceived that ventilation ports 28 (shown in FIG. 1), such as vents or windows, that can be positioned within the outer wall 4 to allow continued ventilation. The ventilation ports 28 can allow for continuous gas flow and can specifically provide continued gas flow if the distal end becomes obstructed. The vents or windows can be oriented on either side of the bronchoscope, although other locations can also be suitable (e.g. dorsal surface, ventral surface, or the like) depending on locations of imaging components. Such vents allow gases to escape if the distal opening of the bronchoscope is obstructed. The vents can have various sizes and shapes, including but not limited to square, rectangular, circular, elliptical, and the like, and can be placed in various locations adjacent the distal end. The number of vents can also be varied and is generally at least two with one each on opposing sides, and in most cases up to about six distributed about the distal end, although any suitable number of vents can be used.

Each of the embodiments contemplated herein can be sterilizable and reusable. Alternatively, the bronchoscope 2 can be disposable depending on the materials used in its manufacture, if made from disposable material and/or if it is not cost prohibitive.

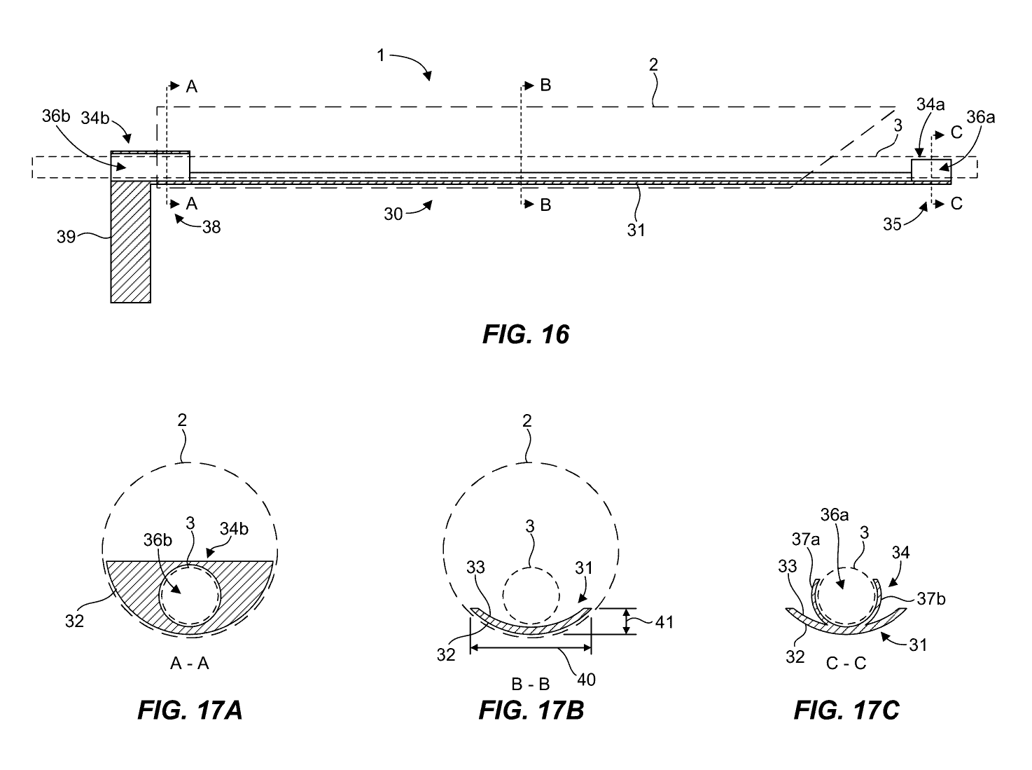

FIG. 16 illustrates a bronchoscope system 1 that can comprise a rigid bronchoscope 2 as disclosed herein (shown in phantom lines to provide context) and one or more bronchoscopy instruments 30. FIGS. 17A-17C illustrate several cross-sectional views of the bronchoscope system 1. The instrument 30 can be used within the bronchoscope 2, which can be designed to improve or maximize the flow of gases during instrumentation as described above. In this case, the instrument 30 is configured as a catheter guide for positioning a catheter 3 (shown in phantom lines to provide context) during a bronchoscopy.

Generally, when configured as rigid bronchoscopy forceps, the instrument can include jaws for grasping, a mechanism for activating the jaws such as levers and hinges, an elongated rigid body that maintains the jaws in a fixed position while allowing the jaws to open and close, and a hand control such as finger rings. In one example, one of the finger rings is fixed to the elongated body and the other finger ring acts on a lever arm.

Three basic configurations of such an instrument are specifically enumerated. In one configuration, a cross-sectional shape would conform closely to the inner wall of the bronchoscope allowing it to move out of the center of the main chamber lumen. In another configuration, a hollow tube can further be attached to the first configuration allowing simultaneous suctioning and forceps instrumentation without having to exchange instruments. In a third configuration, the elongated body can be eliminated and the jaws of the forceps are integrally fixed at the distal tip of the bronchoscope. A thin lever arm can run a length of the bronchoscope acting on the jaws to open and close. The lever arm can be made as small as possible to retain function while minimizing occupied space, and would not necessarily have a cross-sectional shape that conforms to the inner wall of the bronchoscope. The hand control acting on the lever arm can be varied such as standard finger rings, nobs, etc., although other hand control features can be used.

Referring again to FIG. 16, one example of the instrument 30 can include an elongate structure 31, such as a shaft, configured to extend through the bronchoscope 2 (e.g., the working channel 10 described herein). The elongate structure 31 can have a bottom outer surface 32 and a top outer surface 33 opposite the bottom outer surface 32. A catheter coupling feature 34a can be associated with the top outer surface 33 to engage and couple with the catheter 3. The catheter coupling feature 34a can be disposed at a distal end 35 of the elongate structure 31. The catheter coupling feature 34a can have an opening 36a configured to receive at least a portion of the catheter 3. In one aspect, the opening 36a can be defined at least in part by extension members 37a, 37b extending from the top outer surface 33 (see FIG. 17C). The extension members 37a, 37b can be configured to receive the catheter 3 in a snap-in manner. Alternatively, the catheter coupling feature 34a can have an opening that surrounds the catheter 3. In some embodiments, a catheter coupling feature 34b can be associated with a proximal end 38 of the elongate structure 31. The catheter coupling feature 34b can have an opening 36b configured to receive the catheter 3 (see FIG. 17A). The opening 36b can be configured to surround the catheter 3.

Alternatively, the catheter coupling feature 34b can be configured similar to the catheter coupling feature 34a to receive the catheter 3 in a snap-in manner. A handle 39 can associated with the proximal end 38 of the elongate structure 31 to facilitate manipulation of the instrument 30 by a user.

In yet another optional configuration, a hollow tube can be fixed to the forceps. The hollow tube can allow passage of catheters, such as suction catheters, catheter instruments, or catheters with imaging capability. The hollow tube can also be rigid or collapsible to occupy less space when catheters are not in use. Regardless of the specific configuration, the system of the present invention can allow use a forceps and suction catheter simultaneously. The hollow tube or sheath can allow direction of the catheter and some directional control when manipulating the forceps. The tube can also prevent the catheters and forceps from interfering with each other when trying to pass in a restricted space. The catheter can easily slide inside the sheath when suctioning is needed and then be pulled back.

The catheter can be operated in any suitable manner to suction gas or material from a patient's airway. For example, a high-power suction can be applied to evacuate smoke in a constricted space and/or suction can be paced during expiration to limit inhalation of smoke and avoid impairment of ventilation due to suctioning of vital gases.

In one aspect, the outer surface 32 of the elongate structure 31 can be shaped to conform to an inner surface of the working channel (e.g., the inner surface 5 of the working channel 10). Although the elongate structure 31 is illustrated along a bottom surface of the inner surface, such can also be oriented alongside or top surfaces of the inner surface. The elongate structure 31 can therefore occupy a lateral or off-center position (e.g., lumen) of the bronchoscope, rather than a central position. This lateral position can decrease turbulence and resistance and therefore improve the flow of gases through the bronchoscope. This position can also locate the tip of the instrument 30 at an optimal viewing angle of a distal camera of a bronchoscope as described herein. The catheter 3 can be allowed to float freely within the bronchoscope 2 to reduce resistance to gas flow (see FIG. 17B). Alternatively, a hollow tube can extend an entire length of the forceps and can be permanently fixed to the forceps. In this manner, a flexible suction catheter or other device can be inserted inside the hollow tube which acts as a guide channel.

As shown in FIGS. 17B and 17C, a cross-sectional shape of the bottom outer surface 32 perpendicular to the length of the elongate structure 31 can be any suitable shape to conform to or contour an interfacing inner surface of the bronchoscope 2, such as an elliptical shape and/or a lenticular shape. A cross-sectional shape of the top outer surface 33 perpendicular to the length perpendicular to the length of the elongate structure 31 can be any suitable shape, such as a concave shape (as illustrated) and/or a flat shape. Generally, the shape of the elongate structure 31 can provide for efficient flow of gas about the elongate structure within the bronchoscope 2 while providing sufficient structural rigidity. The bottom outer surface 32 can at least partially define a width 40 and a thickness 41 of the elongate structure 31. In one aspect, a ratio of the width 40 to the thickness 41 can be is greater than 1. In other words, the width 40 is greater than the thickness 41. A shape resulting from such dimensions can facilitate positioning the elongate structure 31 in a lateral or off-center position within the bronchoscope 2. The instrument 30 can be free or separate from the bronchoscope 2, integral or connected to an interior wall of the bronchoscope, or removably attachable to the bronchoscope.

In a broad interpretation, the present bronchoscope system establishes an airway while evaluating and treating conditions of the tracheobronchial tree. For example, the bronchoscope and instruments disclosed herein can be used to remove a foreign body from the upper airway, or employed during biopsy and minimally invasive interventions of the upper airway.

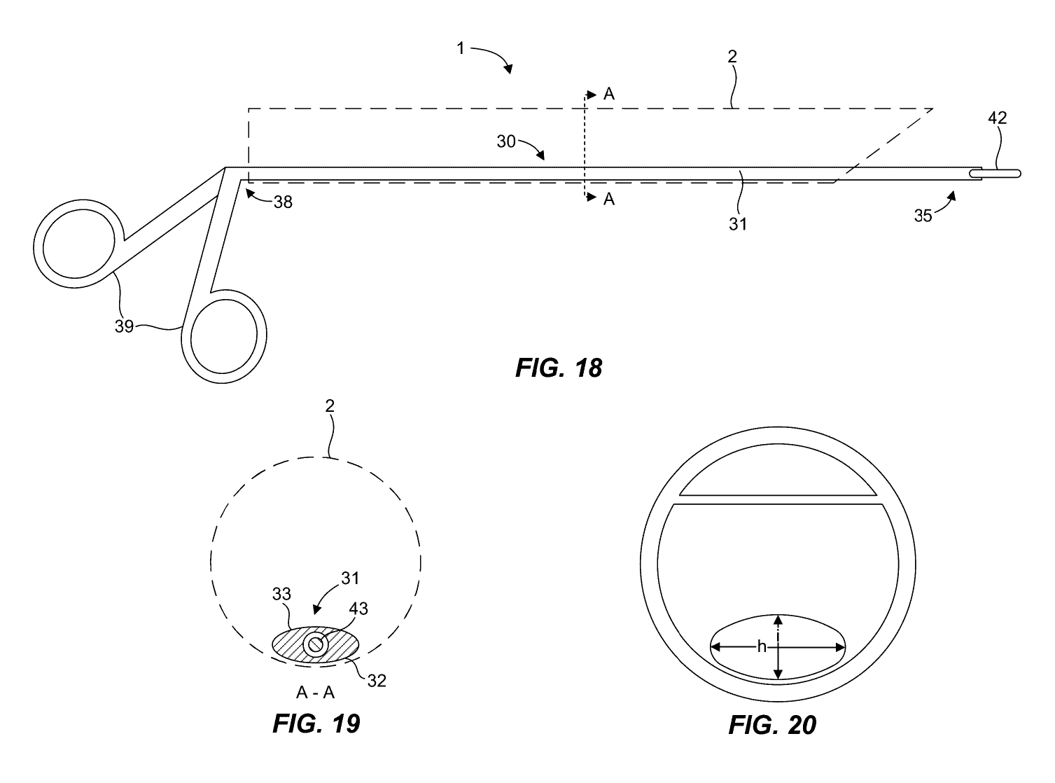

FIG. 18 illustrates another embodiment of a bronchoscope system 1 that can comprise a rigid bronchoscope 2 as disclosed herein (shown in phantom lines to provide context) and one or more bronchoscopy instruments 30. The bronchoscopy instruments can include bronchoscopy forceps or other novel devices as described herein. However, standard forceps can also be used in connection with the rigid airway bronchoscopes herein. FIG. 19 illustrates a cross-sectional view of the bronchoscope system 1. In this case, the instrument 30 is configured as forceps. For example, the instrument 30 can include a plurality of jaws 42 extending from the distal end 35 of the elongate structure 31, and handles 39 at the proximal end 38 to actuate the jaws. An actuation coupling member 43 can be disposed inside the elongate structure 31 to couple the handles 39 to the jaws 42 (see FIG. 19).

As discussed above, the bottom outer surface 32 of the elongate structure 31 can be shaped to conform to or contour an inner surface of the working channel (e.g., the inner surface 5 of the working channel 10). The elongate structure 31 can therefore occupy a lateral or off-center position (e.g., lumen) of the bronchoscope, rather than a central position. This lateral position can improve the flow of gases through the bronchoscope. This position can also locate the jaws 42 of the instrument 30 at an optimal viewing angle of a distal camera of a bronchoscope as described herein.

As shown in FIG. 19, a cross-sectional shape of the bottom outer surface 32 perpendicular to the length of the elongate structure 31 can be any suitable shape to conform to or contour an interfacing inner surface of the bronchoscope 2, such as an elliptical shape and/or a lenticular shape. A cross-sectional shape of the top outer surface 33 perpendicular to the length perpendicular to the length of the elongate structure 31 can be any suitable shape, such as a convex shape (as illustrated). Generally, the shape of the elongate structure 31 can provide for efficient flow of gas about the elongate structure within the bronchoscope 2 while providing sufficient structural rigidity (e.g., laterally stiff yet vertically flexible to allow insertion through a beveled distal opening of a bronchoscope). The bottom outer surface 32 can at least partially define a width and a thickness of the elongate structure 31 (identified by “h” and “i,” respectively, in FIG. 20). In one aspect, a ratio of the width to the thickness can be is greater than 1. In other words, the width is greater than the thickness. A shape resulting from such dimensions can facilitate positioning the elongate structure 31 in a lateral or off-center position within the bronchoscope 2. The instrument 30 can be free or separate from the bronchoscope 2, integral or connected to an interior wall of the bronchoscope, or removably attachable to the bronchoscope. FIG. 20 illustrates a cross-section of an instrument within a bronchoscope, with width and thickness dimensions identified for the elongate structure. Examples of suitable values for these dimensions are found in Table 2, below.

TABLE 2

| | | <30 Weeks | Newborn/ | 0-12 | 12-24 | 2-4 | >4 |
|---|---|---|---|---|---|---|---|
| ID | Description | Premature | Premature | Months | Months | Years | Years |
| h | Ideal Forceps Width (mm) | 1.5 | 2.0 | 2.5 | 3.0 | 3.5 | 4.0 |
| h | Forceps Width (mm) | 0.1-3.5 | 0.1-3.9 | 0.1-4.9 | 0.1-5.6 | 0.1-6.3 | 0.1-6.7 |
| i | Ideal Forceps Height (mm) | 1 | 1.5 | 2.0 | 2.5 | 2.5 | 2.5 |
| i | Forceps Height (mm) | 0.1-3.3 | 0.1-3.7 | 0.1-4.7 | 0.1-5.3 | 0.1-6.1 | 0.1-6.4 |

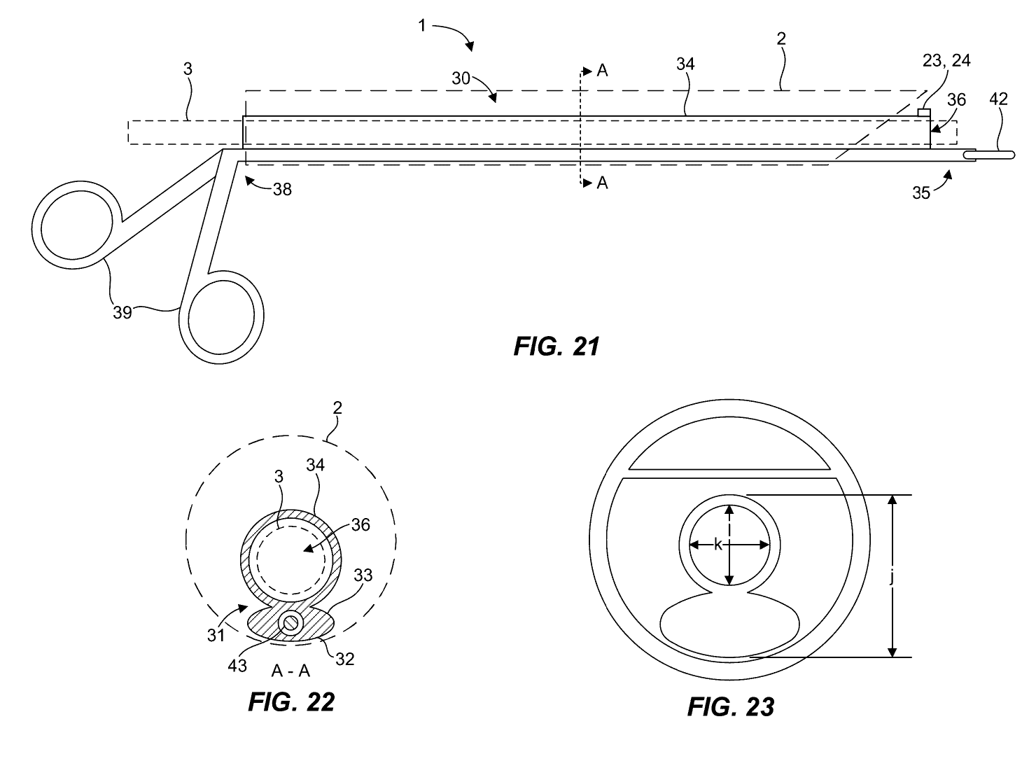

FIG. 21 illustrates another embodiment of a bronchoscope system 1 that can comprise a rigid bronchoscope 2 as disclosed herein (shown in phantom lines to provide context) and one or more bronchoscopy instruments 30. FIG. 22 illustrates a cross-sectional view of the bronchoscope system 1. In this case, the instrument 30 is configured as a catheter forceps, which combines forceps with the features of a catheter guide for positioning a catheter 3 (shown in phantom lines to provide context) during a bronchoscopy to facilitate simultaneous instrumentation and suctioning.

For example, the instrument 30 can include jaws 42 extending from the distal end 35 of the elongate structure 31, and handles 39 at the proximal end 38 to actuate the jaws. The actuation coupling member 43 can be disposed inside the elongate structure 31 to couple the handles 39 to the jaws 42 (see FIG. 22). In addition, the instrument 30 can include the catheter coupling feature 34 associated with the top outer surface 33 to engage and couple with the catheter 3. In some embodiments, the catheter coupling feature 34 can be disposed along the length of the elongate structure 31, as shown in FIG. 21. The catheter coupling feature 34 can have an opening or instrument channel 36 configured to receive the catheter 3. In one aspect, the opening 36 can be configured to surround the catheter 3. For example, the catheter coupling feature can comprise a tube defining the opening 36, and the tube can extend along the length of the elongate structure. Alternatively, one or more openings can be defined at least in part by extension members extending from the top outer surface 33 to receive the catheter 3 in a snap-in manner. In some embodiments, multiple catheter coupling features can be associated with the elongate structure 31. FIG. 23 illustrates a cross-section of an instrument within a bronchoscope, with certain dimensions identified for the instrument. Examples of suitable values for these dimensions are found in Table 3, below.

TABLE 3

| | | <30 Weeks | Newborn/ | 0-12 | 12-24 | 2-4 | >4 |
|---|---|---|---|---|---|---|---|
| ID | Description | Premature | Premature | Months | Months | Years | Years |
| j | Ideal Forceps/Instrument | 1.5 | 2.0 | 3.0 | 3.8 | 4.4 | 4.7 |
| | Channel Max. Dia. (mm) | | | | | | |
| j | Forceps/Instrument | 0.1-3.5 | 0.1-3.9 | 0.1-4.9 | 0.1-5.6 | 0.1-6.3 | 0.1-6.7 |
| | Channel Max. Dia. (mm) | | | | | | |
| k | Ideal Instrument Catheter | 0.8 | 1.5 | 2.1 | 2.8 | 3.4 | 4.1 |
| | Channel I.D. (mm) | | | | | | |
| k | Instrument Channel I.D. | 0.1-3.4 | 0.1-3.8 | 0.1-4.8 | 0.1-5.5 | 0.1-6.2 | 0.1-6.6 |
| | (mm) | | | | | | |
| l | Ideal Instrument Channel | 1 | 1.7 | 2.3 | 3.0 | 3.6 | 4.3 |
| | O.D. (mm) | | | | | | |
| l | Instrument Channel O.D. | 0.1-3.5 | 0.1-3.9 | 0.1-4.9 | 0.1-5.6 | 0.1-6.3 | 0.1-6.7 |
| | (mm) | | | | | | |
| m | Ideal Instrument Channel | 0.1 | 0.1 | 0.1 | 0.1 | 0.1 | 0.1 |
| | Wall Thickness (mm) | | | | | | |
| m | Instrument Channel Wall | 0.1-0.4 | 0.1-0.4 | 0.1-0.4 | 0.1-0.4 | 0.1-0.4 | 0.1-0.4 |
| | Thickness (mm) | | | | | | |

As discussed above, the bottom outer surface 32 of the elongate structure 31 can be shaped to conform to or contour an inner surface of the working channel (e.g., the inner surface 5 of the working channel 10). The elongate structure 31 can therefore occupy a lateral or off-center position (e.g., lumen) of the bronchoscope, rather than a central position. This lateral position can improve the flow of gases through the bronchoscope. This position can also locate the jaws 42 of the instrument 30 at an optimal viewing angle of a distal camera of a bronchoscope as described herein. Regardless, the offset location can be oriented opposite to or adjacent to the imaging system.

As shown in FIG. 22, a cross-sectional shape of the bottom outer surface 32 perpendicular to the length of the elongate structure 31 can be any suitable shape to conform to or contour an interfacing inner surface of the bronchoscope 2, such as an elliptical shape and/or a lenticular shape. A cross-sectional shape of the top outer surface 33 perpendicular to the length perpendicular to the length of the elongate structure 31 can be any suitable shape, such as a convex shape (as illustrated). Generally, the shape of the elongate structure 31 can provide for efficient flow of gas about the elongate structure within the bronchoscope 2 while providing sufficient structural rigidity (e.g., laterally stiff yet vertically flexible to allow insertion through a beveled distal opening of a bronchoscope). The bottom outer surface 32 can at least partially define a width and a thickness of the elongate structure 31. In one aspect, a ratio of the width to the thickness can be is greater than 1. In other words, the width is greater than the thickness. A shape resulting from such dimensions can facilitate positioning the elongate structure 31 in a lateral or off-center position within the bronchoscope 2. The instrument 30 can be free or separate from the bronchoscope 2, integral or connected to an interior wall of the bronchoscope, or removably attachable to the bronchoscope.

In one aspect, an instrument as disclosed herein can include an illumination component (e.g., a light) and/or an imaging component (e.g., a camera). For example, as illustrated in FIG. 21, the instrument 30 can include an illumination component 23 and/or an imaging component 24 at the distal end 35. In this case, the illumination component 23 and/or the imaging component 24 are shown associated with the catheter coupling feature 34, although, in some embodiments, the illumination component 23 and/or the imaging component 24 can be associated with the elongate structure 31. The illumination component 23 and/or the imaging component 24 can be operably coupled to external equipment in any suitable manner disclosed herein, such as utilizing a hardware channel on the instrument 30 and/or wireless technology.

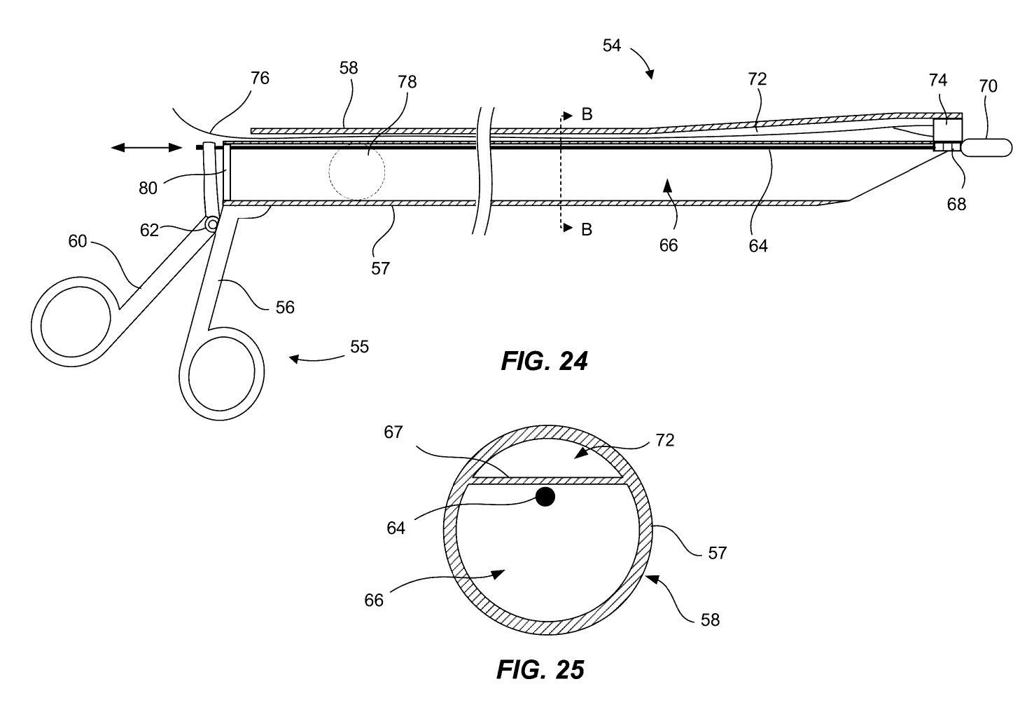

FIG. 24 illustrates a rigid airway bronchoscope system 54 having an integrated forceps 55. In this manner, a separate elongate forceps support shaft can be eliminated. A forcep handle 56 can be fixed to an outer wall 57 of the rigid bronchoscope tube 58. A complimentary forcep actuator lever 60 can be associated with the fixed handle 56 via a hinge 62. A distal end of the actuator lever 60 can be connected to a proximal end of a forcep arm 64 which extends longitudinally within the working channel 66 along an inner wall 67. A distal end of the forcep arm 64 is connected to a jaw activator 68. The jaw activator 68 can be fixed to the rigid tube either at the outer wall 57 or the inner wall 67. The jaw activator 68 is a hinge and lever mechanism which activates jaw 70 to allow grasping of objects by the jaw. As the forcep actuator lever 60 is engaged toward the fixed handle 56, the forcep arm moves longitudinally to engage the jaw activator 68. Thus, the forcep arm 64 is exposed within the working channel 66 which allows for reduced obstruction of air flow and further reduction of potential complications to a patient. Thus, the forcep arm 64 is at least partially supported by the rigid tube walls rather than a dedicated forcep shaft. The forcep arm 64 can be formed of a sufficiently rigid material to allow engagement of the jaw activator 68. Non-limiting examples of suitable material can include surgical steel rod, polycarbonate rod, and the like.

The rigid airway bronchoscope system 54 can further include a hardware channel 72 within the rigid tube 58 as in previously described embodiments. FIG. 25 illustrates a cross-sectional view of FIG. 24 across segment B. As shown, the rigid tube 58 has a circumferential outer wall 57 in which an inner conduit is subdivided into the hardware channel 72 and the working channel 66 by inner wall 67. The forcep arm 64 is freely oriented within the working channel.

As with other embodiments, an imaging and illumination component 74 can be oriented at a distal end of the hardware channel 72 adjacent the jaw activator 68. Corresponding fiber optics and wiring 76 can connect the imaging and illumination component 70 to appropriate power source and computing equipment to produce visual images. As with other embodiments, the rigid tube 58 can also include an anesthetic port 78 to allow delivery of anesthetic gas, drugs, or other gases or vapors. Further, a seal 80 can also be oriented at a proximal end of the rigid tube 58 to allow use of the forceps and insertion of additional instruments while minimizing or eliminating ventilation losses as previously described in connection with FIG. 8.

### EXAMPLE

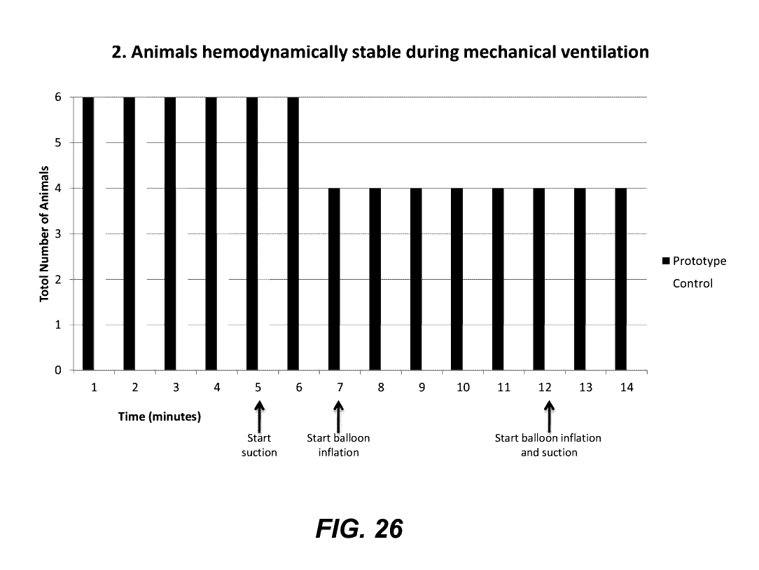

A rigid airway bronchoscope as illustrated in FIGS. 1-2 was fabricated out of stainless steel. The bronchoscope had an inner diameter of 4×5 mm and an outer diameter of 5.7 mm. The airway bronchoscope was inserted into the bronchial tubes of a total of six lambs and a control bronchoscope (Karl Storz, Tuttlingen, Germany) was inserted into the same six lambs in a cross-over study. FIG. 26 is a graph of total number of animals which were hemodynamically stable during mechanical ventilation. In each case, a suction tube was inserted into the bronchoscope with suction and balloon inflation begun at time 5, 7 and 12 minutes, respectively. Notably, after initiation of suction four of the six animals having the airways bronchoscope remained hemodynamically stable throughout the procedure. In contrast, only one of six animals having the traditional bronchoscope remained stable throughout the procedure. Oxygen saturation and heart rate began to decline dramatically in the control group after about 1 minute of mechanical ventilation. These results indicate a strong improvement in ventilation and hemodynamic stability using the inventive airway bronchoscope.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A bronchoscope, comprising:
a rigid tube for insertion into an airway of a patient, the rigid tube having a proximal end, a distal end, and a working channel extending between the proximal and distal ends sized to facilitate ventilation of the airway via gas flow through the working channel;
a gas port at the proximal end of the rigid tube in communication with the working channel to deliver a gas to the airway via the working channel;
an instrument port at the proximal end of the rigid tube in communication with the working channel to facilitate insertion of an instrument through the working channel into the airway; and
at least one of an illumination component and an imaging component at the distal end of the rigid tube.

2. The bronchoscope of claim 1, wherein the gas port is in communication with the working channel such that gas flow is laminar between the gas port and the working channel.

3. The bronchoscope of claim 1, wherein the gas port is oriented at an angle less than 90 degrees relative to a longitudinal axis of the rigid tube and has an inlet curvature sufficient to reduce turbulence.

4. The bronchoscope of claim 1, wherein the rigid tube is beveled at the distal end.

5. The bronchoscope of claim 1, wherein the working channel is configured to maintain laminar flow therethrough.

6. The bronchoscope of claim 1, wherein the instrument port is oriented parallel to a longitudinal axis of the rigid tube.

7. The bronchoscope of claim 1, wherein the rigid tube further comprises a hardware channel, and the at least one of the illumination component and the imaging component is disposed at an end of the hardware channel.

8. The bronchoscope of claim 7, further comprising a hardware port at the proximal end of the rigid tube in communication with the hardware channel to facilitate communication with the at least one of the illumination component and the imaging component.

9. The bronchoscope of claim 7, wherein the working channel and the hardware channel are separated by an inner wall.

10. The bronchoscope of claim 9, wherein the at least one of the illumination component and the imaging component is positioned within an outer wall of the rigid tube.

11. The bronchoscope of claim 10, wherein distal ends of the inner and outer walls are nonparallel in a direction along a longitudinal axis of the rigid tube.

12. The bronchoscope of claim 11, wherein the distal end of the outer wall is angled away from the longitudinal axis.

13. The bronchoscope of claim 11, wherein the distal end of the inner wall is angled toward the longitudinal axis.

14. The bronchoscope of claim 11, wherein the distal end of the inner wall or the distal end of the outer wall is straight in the direction along the longitudinal axis.

15. The bronchoscope of claim 10, wherein distal ends of the inner and outer walls are parallel in a direction along a longitudinal axis of the rigid tube.

16. The bronchoscope of claim 1, wherein the imaging component comprises a camera.

17. The bronchoscope of claim 16, wherein a focal length of the camera and a camera angle is such that a distal tip of the rigid tube is centrally positioned within a tracheobronchial tree when an image is centered at a central lumen of the tracheobronchial tree.

18. The bronchoscope of claim 1, wherein a cross-sectional shape of an inner surface of the working channel is at least partially rounded.

19. The bronchoscope of claim 1, further comprising a seal associated with the instrument port to seal about the instrument when inserting the instrument through the working channel into the airway.

20. The bronchoscope of claim 19, wherein the seal comprises an opening in a flexible membrane.

21. The bronchoscope of claim 20, wherein the opening is defined by a slit in the flexible membrane.

22. The bronchoscope of claim 1, further comprising an integrated forceps wherein the integrated forceps comprises:
a. a forcep actuator attached to the rigid tube at the proximal end;
b. a forcep arm connected to the forcep actuator and extending to the distal end within the working channel;
c. a jaw activator connected to the forcep arm and oriented at the distal end; and
d. a jaw operably engageable by the jaw activator to allow grasping of an object.

23. A bronchoscopy instrument, comprising:
an elongate structure configured to extend through a bronchoscope, the elongate structure having an outer surface at least partially defining a width and a thickness of the elongate structure,
wherein a ratio of the width to the thickness is greater than 1, and the outer surface is shaped to conform to an inner surface of the bronchoscope.

24. The bronchoscopy instrument of claim 23, wherein a cross-sectional shape of the outer surface perpendicular to a length of the elongate structure is elliptical, lenticular, or a combination thereof.

25. The bronchoscopy instrument of claim 23, wherein a cross-sectional shape of the elongate structure perpendicular to a length of the elongate structure is elliptical, lenticular, or a combination thereof.

26. The bronchoscopy instrument of claim 23, further comprising a plurality of jaws extending from a distal end of the elongate structure, wherein the elongate structure forms at least a portion of a forceps.

27. The bronchoscopy instrument of claim 23, further comprising a second outer surface opposite the first outer surface.

28. The bronchoscopy instrument of claim 27, wherein a cross-sectional shape of the second outer surface perpendicular to a length of the elongate structure is convex, concave, or flat.

29. The bronchoscopy instrument of claim 27, further comprising a catheter coupling feature associated with the second outer surface to engage and couple with a catheter.

30. The bronchoscopy instrument of claim 29, further comprising a plurality of jaws extending from a distal end of the elongate structure, wherein the elongate structure forms at least a portion of a forceps.

31. The bronchoscopy instrument of claim 29, wherein the catheter coupling feature is disposed at a distal end of the elongate structure.

32. The bronchoscopy instrument of claim 29, wherein the catheter coupling feature comprises an opening configured to receive at least a portion of the catheter.

33. The bronchoscopy instrument of claim 32, wherein the opening is at least partially defined by first and second extension members extending from the second outer surface.

34. The bronchoscopy instrument of claim 32, wherein the catheter coupling feature comprises a tube defining the opening.

35. The bronchoscopy instrument of claim 34, wherein the tube extends along a length of the elongate structure.

36. The bronchoscopy instrument of claim 23, further comprising at least one of an illumination component and an imaging component at a distal end of the elongate structure.

37. The bronchoscopy instrument of claim 36, wherein the imaging component comprises a camera.

38. A bronchoscope system, comprising:
a bronchoscope including
a rigid tube for insertion into an airway of a patient, the rigid tube having a proximal end, a distal end, and a working channel extending between the proximal and distal ends sized to facilitate ventilation of the airway via gas flow through the working channel,
a gas port at the proximal end of the rigid tube in communication with the working channel to deliver a gas to the airway via the working channel,
an instrument port at the proximal end of the rigid tube in communication with the working channel to facilitate insertion of an instrument through the working channel into the airway, and
at least one of an illumination component and an imaging component at the distal end of the rigid tube; and
a bronchoscopy instrument including
an elongate structure configured to extend through the working channel, the elongate structure having an outer surface at least partially defining a width and a thickness of the elongate structure,
wherein a ratio of the width to the thickness is greater than 1, and the outer surface is shaped to conform to an inner surface of the working channel.
